(19) United States
(12) Patent Application Publication
(10) Pub. No.: US 2007/0027216 A1
(43) Pub. Date: Feb. 1, 2007

(54) NOVEL HYDROCHLORIDE SALTS OF LEVODOPA

(76) Inventors: Bridget Larson, Brookline, MA (US); Anthony Meehan, Shrewsbury, MA (US); Julius Remenar, Framingham, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/457,647

(22) Filed: Jul. 14, 2006

Related U.S. Application Data
(60) Provisional application No. 60/699,882, filed on Jul. 15, 2005.

(51) Int. Cl. A61K 31/198; C07C 229/34

(52) U.S. Cl. 514/567; 562/444

(57) ABSTRACT

The present invention provides a novel hydrochloride salt of levodopa. In addition, pharmaceutical compositions comprising said hydrochloride salt of levodopa may be used as fast-dissolve compositions. Methods of making and of using the same are also provided.

### FIELD OF THE INVENTION

The present invention relates to a novel hydrochloride salt of levodopa. The invention also provides improved methods of using levodopa hydrochloride and improved methods of treatment with levodopa hydrochloride.

### BACKGROUND OF THE INVENTION

Parkinson's disease is a neurodegenerative disorder characterized by a progressive degeneration of the dopaminergic pathway in the brain. Parkinson's patients often have symptoms of bradykinesia, rigidity, tremor, poor balance and difficulty walking. One of the most common treatments for Parkinson's disease is oral administration of levodopa. Levodopa functions to cross the blood brain barrier, converts to dopamine, and replaces or supplements low levels of dopamine in the brain. Parkinson's disease patients often take between 200 mg and 2 g of levodopa in tablet form per day with late stage Parkinson's patients taking toward the latter end of this range. Most levodopa tablets also comprise a dopa decarboxylase inhibitor, such as carbidopa, as in the case of SINEMET® tablets. One of the disadvantages with levodopa/carbidopa tablets is that Parkinson's patients often experience episodes of “wearing off.” During these episodes, patients become frozen or have rigid movements. These freezing episodes have significant detrimental consequences to the quality of life for Parkinson's patients. To recover from a freezing episode, patients often administer levodopa/carbidopa tablets under their tongue rather than swallowing the tablet. Administration of the drug under a patient's tongue will often not release a patient from a frozen episode for an hour. A controlled release tablet version of levodopa/carbidopa (SINEMET® CR) is also available to patients. The controlled release SINEMET® CR has not provided much improved clinical effects. As a result, patients taking SINEMET® CR still have “wearing off” and freezing episodes of significant duration. Thus, a need exists for a fast-dissolve levodopa formulation which may decrease the duration of such freezing episodes.

Levodopa is one isomer of the molecule, dopa, also known as 3-hydroxytyrosine. Dopa is a chiral molecule. Therefore, dopa exists as two isomers, L-dopa and D-dopa. However, it is only the L-dopa (levodopa) isomer, in an isolated form, which has been used extensively for its pharmacological activity to treat conditions such as, but not limited to, Parkinson's disease.

Levodopa (CAS Registry Number: 59-92-7), also known as 3-hydroxy-L-tyrosine, L-dopa, or (−)-2-amino-3-(3,4-dihydroxyphenyl)propanoic acid is represented by structure (I):

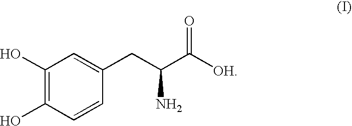

It would be advantageous for Parkinson's patients to have a new form of levodopa that has improved properties, in particular, as oral formulations. Specifically, it is desirable to increase the dissolution rate of levodopa-containing pharmaceutical compositions in water and/or to provide a more rapid onset to therapeutic effect. In addition, it is desirable to provide a salt of levodopa with increased aqueous solubility relative to the known free base form. It is also desirable to have a form of levodopa which, when administered to a subject, reaches a peak plasma level faster and/or has a longer lasting plasma concentration and higher overall exposure at high doses when compared to equivalent amounts of levodopa in its known free base form.

### SUMMARY OF THE INVENTION

It has now been found that a novel hydrochloride salt of levodopa can be obtained which has improved properties as compared to the known free base form of levodopa.

Accordingly, in a first aspect, the present invention provides a novel hydrochloride salt of levodopa.

The invention further provides pharmaceutical compositions comprising a hydrochloride salt of levodopa, methods of making the hydrochloride salt of levodopa, and related methods of treatment.

In a further aspect, the present invention provides a process for the preparation of a hydrochloride salt of levodopa, which comprises mixing levodopa with hydrochloric acid to form a mixture and allowing for precipitation of said hydrochloride salt of levodopa to occur.

In a still further aspect of the invention, a method is provided for treating a mammal suffering from a condition, such as Parkinson's disease, which comprises administering to the mammal an effective amount of a hydrochloride salt of levodopa.

The invention further provides a medicament comprising a hydrochloride salt of levodopa and methods of making the same. Typically, the medicament further comprises one or more pharmaceutically-acceptable carriers, diluents or excipients. Medicaments according to the invention are described in further detail below.

### BRIEF DESCRIPTION OF THE DRAWINGS

FIG. 1—PXRD diffractogram of levodopa hydrochloride salt.

FIG. 2—DSC thermogram of levodopa hydrochloride salt.

FIG. 3—TGA thermogram of levodopa hydrochloride salt.

FIG. 4—Raman spectrum of levodopa hydrochloride salt.

FIG. 5—In vitro dissolution data of levodopa hydrochloride salt and levodopa free base.

### DETAILED DESCRIPTION OF THE INVENTION

The present invention relates to a novel hydrochloride salt of levodopa. The hydrochloride salt of levodopa has improved dissolution properties with respect to the known free base form and thus is an advantageous salt for making a fast-dissolve formulation of levodopa. The hydrochloride salt may take several forms including, but not limited to, hydrates and solvates as well as various stoichiometric ratios of levodopa to hydrochloric acid. The invention also includes other forms of levodopa hydrochloride salt including, but not limited to, polymorphs, co-crystals, and amorphous forms of the salt. The invention also provides novel pharmaceutical compositions comprising these forms, methods of making these forms, and related methods of treatment.

In a first embodiment, the present invention comprises levodopa hydrochloride salt.

In a further embodiment, the hydrochloride salt of levodopa can be incorporated into a fast-dissolve formulation. In a specific embodiment, said formulation is a solid oral dosage form. This improvement is partly due to the fact that levodopa hydrochloride salt has enhanced dissolution properties relative to the known levodopa free base. Levodopa hydrochloride salt may enable a faster onset of action for such a fast-dissolve formulation.

In another embodiment, the present invention provides a levodopa salt suitable for an oral dosage form. Such a form can be prepared, for example, as a pharmaceutical composition.

In another embodiment, the present invention provides a novel salt form of levodopa with improved solubility relative to the known free base form of levodopa. In a specific embodiment, the present invention provides a novel salt form of levodopa with improved solubility suitable for a pharmaceutical composition. In another specific embodiment, the present invention provides a novel salt form of levodopa with improved solubility suitable for an oral dosage form, such as a fast-dissolve dosage form.

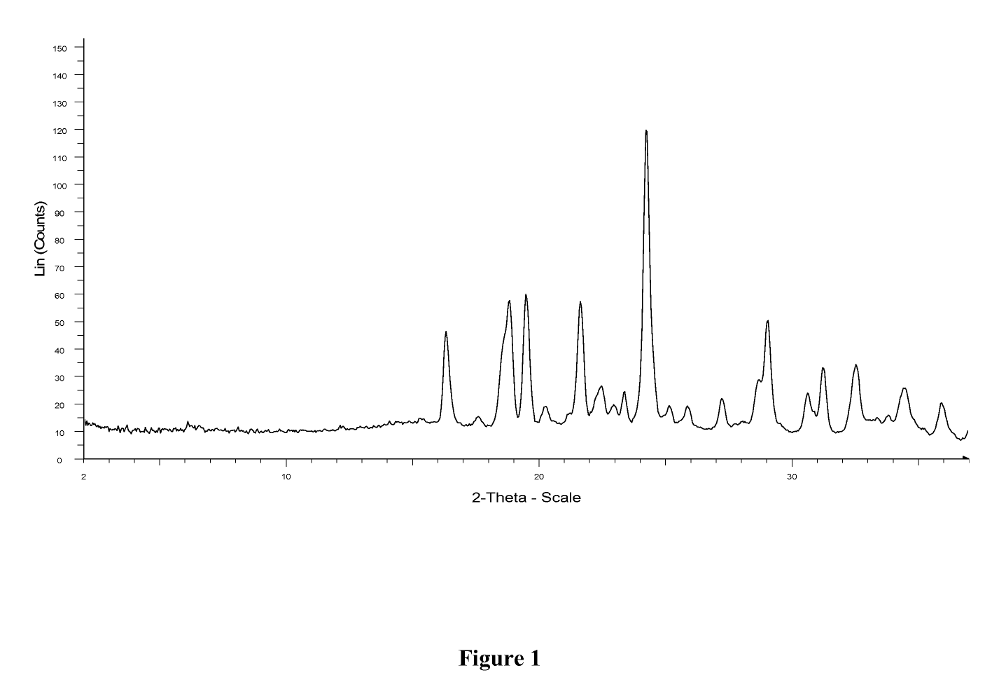

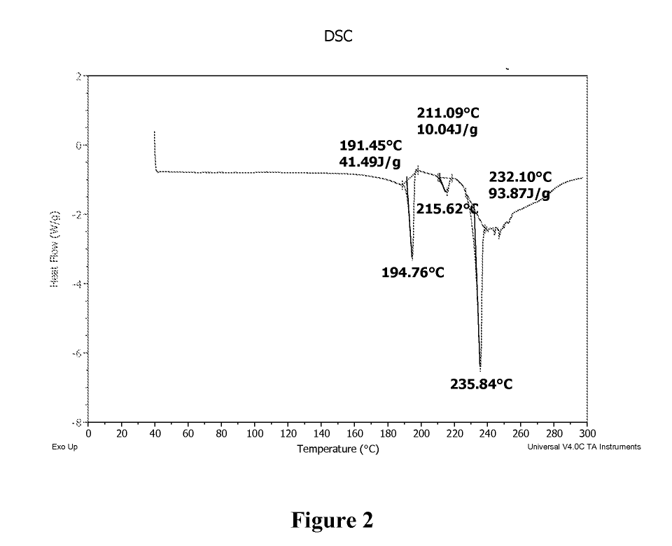

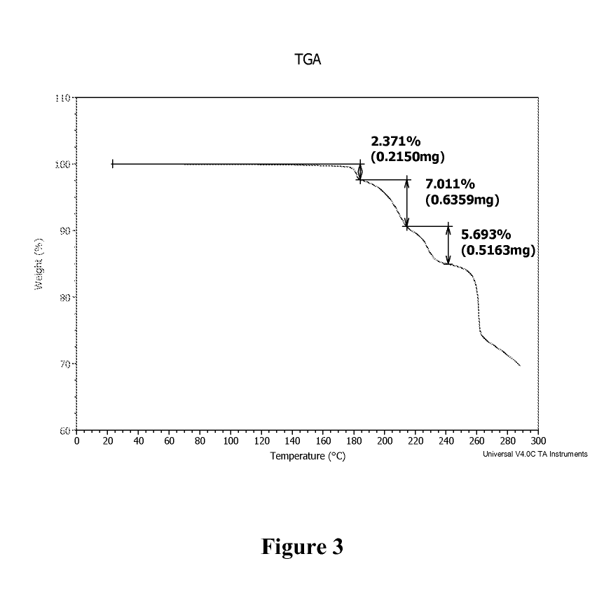

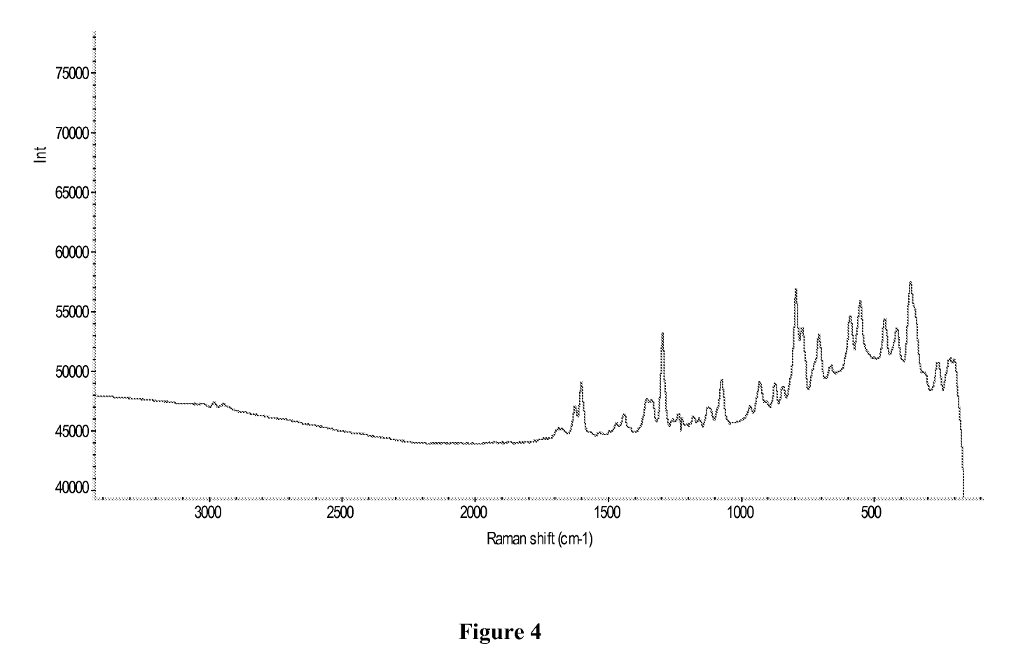

In another embodiment, the present invention comprises levodopa hydrochloride salt characterized by a PXRD diffractogram peak at about 16.32 degrees 2-theta. In another embodiment, the present invention comprises levodopa hydrochloride salt characterized by a PXRD diffractogram peak at about 21.65 degrees 2-theta. In another embodiment, the present invention comprises levodopa hydrochloride salt characterized by a PXRD diffractogram peak at about 24.25 degrees 2-theta. In another embodiment, the present invention comprises levodopa hydrochloride salt characterized by PXRD diffractogram peaks at about 16.32 and about 21.65 degrees 2-theta. In another embodiment, the present invention comprises levodopa hydrochloride salt characterized by PXRD diffractogram peaks at about 16.32 and about 24.25 degrees 2-theta. In another embodiment, the present invention comprises levodopa hydrochloride salt characterized by PXRD diffractogram peaks at about 18.82 and about 19.51 degrees 2-theta. In another embodiment, the present invention comprises levodopa hydrochloride salt characterized by PXRD diffractogram peaks at about 16.32 and about 29.05 degrees 2-theta. In another embodiment, the present invention comprises levodopa hydrochloride salt characterized by PXRD diffractogram peaks at about 16.32, about 21.65, and about 24.25 degrees 2-theta. In another embodiment, the present invention comprises levodopa hydrochloride salt characterized by PXRD diffractogram peaks at about 16.32, about 18.82, and about 19.51 degrees 2-theta. In another embodiment, the present invention comprises levodopa hydrochloride salt characterized by PXRD diffractogram peaks at about 18.82, about 19.51, and about 24.25 degrees 2-theta. In another embodiment, the present invention comprises levodopa hydrochloride salt characterized by PXRD diffractogram peaks at about 21.65, about 27.27, and about 29.05 degrees 2-theta. In another embodiment, the present invention comprises levodopa hydrochloride salt characterized by PXRD diffractogram peaks at about 16.32, about 18.82, about 19.51, about 21.65, and about 24.25 degrees 2-theta. In another embodiment, the present invention comprises levodopa hydrochloride salt characterized by PXRD diffractogram peaks at about 16.32, about 18.82, about 19.51, about 25.90, and about 29.05 degrees 2-theta. In another embodiment, the present invention comprises levodopa hydrochloride salt characterized by PXRD diffractogram peaks at about 16.32, about 21.65, about 24.25, about 27.27, about 29.05 and about 31.26 degrees 2-theta. In another embodiment, the present invention comprises levodopa hydrochloride salt characterized by a PXRD diffractogram substantially similar to FIG. 1. In another embodiment, the present invention comprises levodopa hydrochloride salt characterized by a DSC thermogram substantially similar to FIG. 2. In another embodiment, the present invention comprises levodopa hydrochloride salt characterized by a TGA thermogram substantially similar to FIG. 3. In another embodiment, the present invention comprises levodopa hydrochloride salt characterized by a Raman spectrum substantially similar to FIG. 4.

In another embodiment, the present invention comprises levodopa hydrochloride salt, and methods of making and using the same. In another embodiment, the present invention comprises a solvate of levodopa hydrochloride salt, and methods of making and using the same. In another embodiment, the present invention comprises a hydrate of levodopa hydrochloride salt, and methods of making and using the same. In another embodiment, the present invention comprises one or more polymorphs of levodopa hydrochloride salt or one or more polymorphs of a hydrate or a solvate of levodopa hydrochloride salt, and methods of making and using the same. In another embodiment, the present invention comprises a co-crystal of levodopa hydrochloride salt and a pharmaceutically acceptable co-crystal former, and methods of making and using the same. In another embodiment, the present invention comprises a co-crystal of levodopa hydrochloride salt and a co-crystal former, and methods of making and using the same. In another embodiment, the present invention comprises an amorphous form of levodopa hydrochloride salt, and methods of making and using the same.

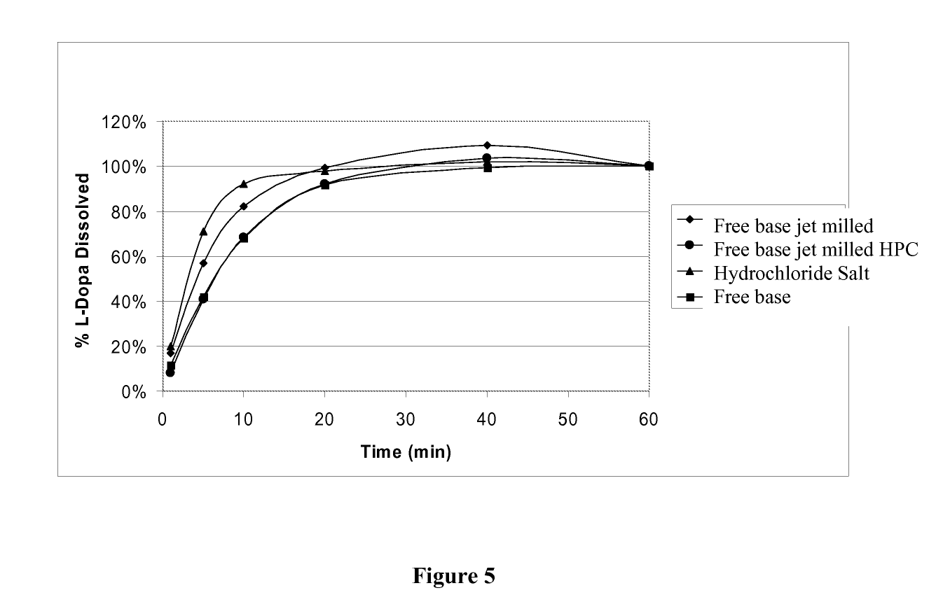

In another embodiment, as illustrated in FIG. 5, a levodopa hydrochloride salt of the present invention reaches 90 percent in vitro dissolution (of levodopa salt) at least about 1.25 times faster than that of levodopa free base. In another embodiment, as illustrated in FIG. 5, a levodopa hydrochloride salt of the present invention reaches 90 percent in vitro dissolution (of levodopa salt) at least about 1.5 times faster than that of levodopa free base. In another embodiment, as illustrated in FIG. 5, a levodopa hydrochloride salt of the present invention reaches 90 percent in vitro dissolution (of levodopa salt) at least about 2.0 times faster than that of levodopa free base. In another embodiment, as illustrated in FIG. 5, a levodopa hydrochloride salt of the present invention reaches 90 percent in vitro dissolution (of levodopa salt) at least about 2.5 times faster than that of levodopa free base. Because of the in vitro dissolution enhancement of levodopa hydrochloride with respect to the known free base form, as shown in Example 2, it is expected that levodopa hydrochloride will also exhibit improved dissolution properties in vivo.

In another embodiment, the present invention comprises levodopa hydrochloride salt in a pharmaceutical composition. In another embodiment, the present invention comprises the combination of levodopa hydrochloride salt and a dopa decarboxylase inhibitor in a pharmaceutical composition. In another embodiment, the present invention comprises the combination of levodopa hydrochloride salt and carbidopa in a pharmaceutical composition.

In another embodiment, the present invention comprises levodopa hydrochloride salt in a powder form (or a powder formulation). Such a powder form may be used to prepare a liquid oral dosage form of the levodopa salt in a similar manner as that known in the art for other known levodopa forms. For example, liquid oral dosage forms are described in US Published Application Nos. US20050070608 and US20050203185, both of which are herein incorporated by reference. The incorporation of levodopa hydrochloride salt into such liquid oral formulations may facilitate the dissolution process and lead to a liquid dosage form that is prepared more easily than that of the known free base form.

In another embodiment, the present invention comprises the combination of levodopa hydrochloride salt and another form of levodopa in a mixture. In another embodiment, the present invention comprises the combination of levodopa hydrochloride salt and the known free base form in a mixture. The present invention also comprises pharmaceutical compositions comprising the combination of levodopa hydrochloride salt and another form of levodopa, such as the known free base form. For example, the present invention includes pharmaceutical compositions comprising the combination of levodopa hydrochloride and another form of levodopa where the total amount of the salt is, for example, greater than about 99 percent, about 95 percent, about 90 percent, about 85 percent, about 80 percent, about 75 percent, about 70 percent, about 65 percent, about 60 percent, about 55 percent, about 50 percent, about 45 percent, about 40 percent, about 35 percent, about 30 percent, about 25 percent, about 20 percent, about 15 percent, about 10 percent, about 5 percent, or between 5 percent and 0.0 percent (exclusive), of the total amount of all levodopa forms present.

In a further aspect, the present invention provides a process for the preparation of a hydrochloride salt of levodopa, which comprises mixing levodopa with hydrochloric acid to form a mixture and allowing for precipitation of said hydrochloride salt of levodopa to occur.

In one embodiment, the levodopa may be mixed with the hydrochloric acid in solution. Any conventional suitable solvent may be used, including organic solvents or mixed solvents. For example, a solvent such as acetonitrile may be used.

Any conventional conditions which salify the levodopa from solution may be used whereby crystals of the levodopa salt are formed. Conveniently, this includes evaporation of the solvent so as to concentrate the solute whereby levodopa salt crystals may be precipitated. In another embodiment, the solution is first heated to ensure mixing and salt formation, followed by cooling so as to buttress the precipitation of salt crystals.

The salt, typically in the form of crystals, may be isolated by any conventional techniques.

The amount of hydrochloric acid used to make a levodopa hydrochloride salt is typically about 1.0-1.5 equivalents of hydrochloric acid for each equivalent of levodopa. These mole ratios can be found when a levodopa hydrochloride salt is prepared according to methods described herein. Other mole ratios can also be used in various methods. The physical form of the levodopa hydrochloride salt is, optionally, compatible with its ability to be formulated into a pharmaceutical composition readily. In one embodiment, the levodopa hydrochloride salt is in a crystalline form and such crystalline forms are readily preparable according to the methods described herein.

Levodopa free base can be prepared by one or more methods available in the art, including, but not limited to, the method in U.S. Pat. No. 3,253,023 or U.S. Pat. No. 3,405,159.

In a further aspect, the present invention provides a process for modulating the solubility of levodopa, which process comprises mixing levodopa with hydrochloric acid to form a mixture and allowing for precipitation of said hydrochloride salt of levodopa to occur.

In one embodiment, the process for modulating the solubility of levodopa is used for the preparation of a pharmaceutical composition.

In one embodiment of the present invention, an amount of levodopa hydrochloride salt effective to treat a mammal is administered to said mammal.

In another embodiment, a method of treating Parkinson's disease is provided, comprising administering an effective amount of levodopa hydrochloride salt to a mammal in need thereof. In another embodiment, said mammal is a human.

In another embodiment, the present invention includes the preparation of a medicament comprising a hydrochloride salt of levodopa. Such a medicament can be used for treating Parkinson's disease, in a mammal in need of such treatment. In another embodiment, said mammal is a human.

In other embodiments, the levodopa hydrochloride salt of the present invention may also be used to treat disorders, such as senile dementia, dementia of the Alzheimer's type, a memory disorder, depression, hyperactive syndrome, a neurodegenerative disease, a neurotoxic injury, brain ischemia, a head trauma injury, a spinal trauma injury, schizophrenia, attention deficit disorder, multiple sclerosis, withdrawal symptoms, epilepsy, convulsions, or seizures, where levodopa is an effective active pharmaceutical for said disorder.

In another embodiment, a method of treating senile dementia, dementia of the Alzheimer's type, a memory disorder, depression, hyperactive syndrome, a neurodegenerative disease, a neurotoxic injury, brain ischemia, a head trauma injury, a spinal trauma injury, schizophrenia, attention deficit disorder, multiple sclerosis, withdrawal symptoms, epilepsy, convulsions, or seizures is provided, comprising administering an effective amount of levodopa hydrochloride salt to a mammal in need thereof. In another embodiment, said mammal is a human.

Levodopa hydrochloride salt can be administered using many known pharmaceutical dosage forms including, but not limited to, oral administration. While levodopa hydrochloride has particular advantages for fast-dissolve oral formulations, the levodopa hydrochloride salt of the present invention may also be used to prepare pharmaceutical dosage forms other than oral dosage forms, such as topical dosage forms, parenteral dosage forms, transdermal dosage forms, and mucosal dosage forms. For example, such forms include creams, lotions, solutions, suspensions, emulsions, ointments, powders, patches, suppositories, and the like. Oral pharmaceutical compositions and dosage forms are exemplary dosage forms. Optionally, the oral dosage form is a solid dosage form, such as a tablet, a caplet, a hard gelatin capsule, a starch capsule, a hydroxypropyl methylcellulose (HPMC) capsule, or a soft elastic gelatin capsule. Liquid dosage forms may also be provided by the present invention, including such non-limiting examples as a suspension, a solution, syrup, or an emulsion.

In another embodiment, the levodopa hydrochloride salt can be incorporated into an osmotically active formulation suitable for oral administration. Osmotically active formulations, osmotic pumps, osmotic drug delivery, and other osmotic technology suitable for oral administration can include, but are not limited to, OROS® Push-Pull and OROS® Tri-layer formulations.

Levodopa hydrochloride salt can be administered by controlled- or delayed-release means. Controlled-release pharmaceutical products generally have a common goal of improving drug therapy over that achieved by their non-controlled release counterparts. Ideally, the use of an optimally designed controlled-release preparation in medical treatment is characterized by a minimum of API substance being employed to cure or control the condition in a minimum amount of time. Advantages of controlled-release formulations generally include: 1) extended activity of the API; 2) reduced dosage frequency; 3) increased patient compliance; 4) usage of less total API; 5) reduction in local or systemic side effects; 6) minimization of API accumulation; 7) reduction in blood level fluctuations; 8) improvement in efficacy of treatment; 9) reduction of potentiation or loss of API activity; and 10) improvement in speed of control of diseases or conditions. (Kim, Cherng-ju, Controlled Release Dosage Form Design, 2 Technomic Publishing, Lancaster, Pa.: 2000).

Like the amounts and types of excipients, the amounts and specific type of active ingredient in a dosage form may differ depending on factors such as, but not limited to, the route by which it is to be administered to mammals. However, typical daily dosage forms of the invention comprise levodopa hydrochloride salt, in an amount of from about 50.0 mg to about 500.0 mg, from about 75.0 mg to 250.0 mg, or from about 100.0 mg to about 250.0 mg. In a particular embodiment, the levodopa hydrochloride salt for use in such a composition is levodopa hydrochloride salt as described herein. Typical daily dosages of the invention comprise levodopa hydrochloride salt, in an amount of from about 50.0 mg to about 2000.0 mg, from about 100.0 mg to about 2000.0 mg, or from about 250.0 mg to about 2000.0 mg. The dosage amounts described herein are expressed in amounts of levodopa free base and do not include the weight of a counterion (e.g., hydrochloride) or any water or solvent molecules.

In another embodiment of the invention, a pharmaceutical composition comprising levodopa hydrochloride salt is administered orally as needed in an amount of from about 50.0 mg to about 500.0 mg, or from about 100.0 mg to about 250.0 mg. For example, about 50.0, 60.0, 70.0, 80.0, 90.0, 100.0, 110.0, 120.0, 130.0, 140.0, 150.0, 160.0, 170.0, 180.0, 190.0, 200.0, 210.0, 220.0, 230.0, 240.0, 250.0, 260.0, 270.0, 280.0, 290.0, or 300.0 mg. In specific embodiments, pharmaceutical compositions comprising levodopa hydrochloride salt can be administered orally in amounts of about 50.00 mg or about 100.0 mg or about 150.0 mg or about 200.0 mg or about 250.0 mg. The dosage amounts can be administered in single or divided doses. In another embodiment, a daily dose of a pharmaceutical composition comprising levodopa hydrochloride salt comprises up to about 2000.0 mg levodopa. In other embodiments, the present invention is directed to compositions comprising levodopa hydrochloride salt as described herein and one or more diluents, carriers, and/or excipients suitable for the administration to a mammal for the treatment or prevention of one or more of the conditions described herein. In one embodiment, a fast-dissolve formulation of levodopa hydrochloride requires a less complex mixture of excipients than other formulations.

The levodopa hydrochloride salt forms of the present invention can be characterized, e.g., by the TGA or DSC data, or by any one, any two, any three, any four, any five, any six, any seven, any eight, any nine, any ten, or any single integer number of Raman peaks or PXRD 2-theta angle peaks, or by any combination of the data acquired from the analytical techniques described above.

Although the invention has been described with respect to various embodiments, it should be realized this invention is also capable of a wide variety of further and other embodiments within the spirit and scope of the appended claims.

### EXAMPLES

### Example 1

### Levodopa Hydrochloride [(−)-2-amino-3-(3,4-dihydroxyphenyl)propanoic acid hydrochloride]

To a slurry containing levodopa (15.1 g, 0.0766 mol) in acetonitrile (750 ml) was added concentrated hydrochloric acid (7.0 mL, 0.0781 mol) dropwise with stirring. Addition of hydrochloric acid to the slurry caused it to form a clear solution followed by precipitation of levodopa hydrochloride. The solid material was collected by filtration and dried under a flow of nitrogen overnight. The solid was characterized using PXRD (Bruker), DSC, TGA and Raman spectroscopy. The aqueous solubility of levodopa hydrochloride salt was determined to be about 11-16 mg/mL at about 25 degrees C. The aqueous solubility of the free base is about 1.65 mg/mL.

The levodopa hydrochloride salt can be characterized by any one, any two, any three, any four, any five, or any six or more of the x-ray diffraction peaks in FIG. 1 including, but not limited to, 16.32, 18.82, 19.51, 21.65, 24.25, 27.27, 29.05, 31.26, and 32.57 degrees 2-theta (as collected). DSC showed two endothermic transitions at about 195 and about 236 degrees C. (See FIG. 2). TGA showed about a 15 percent weight loss from about room temperature to about 240 degrees C. (See FIG. 3). The levodopa hydrochloride salt can be characterized by any one, any two, any three, any four, any five, or any six or more of the Raman shift peaks in FIG. 4 including, but not limited to, 1601, 1295, 1073, 930, 794, 708, 591, 553, 460, 415, and 364 cm⁻¹.

The novel salts of this invention were characterized using the following known techniques and equipment:

### Differential Scanning Calorimetry

DSC analysis of each sample was performed using a Q 1000 Differential Scanning Calorimeter (TA Instruments, New Castle, Del., U.S.A.), which uses Advantage for QW-Series, version 1.0.0.78, Thermal Advantage Release 2.0 (©2001 TA Instruments-Water LLC), with the following components: QDdv.exe version 1.0.0.78 build 78.2; RHBASE.DLL version 1.0.0.78 build 78.2; RHCOMM.DLL version 1.0.0.78 build 78.0; RHDLL.DLL version 1.0.0.78 build 78.1; an TGA.DLL version 1.0.0.78 build 78.1. In addition, the analysis software used was Universal Analysis 2000 for Windows 95/95/2000/NT, version 3.1E; Build 3.1.0.40 (©2001 TA Instruments-Water LLC).

For all of the DSC analyses, an aliquot of a sample was weighed into either a standard aluminum pan (Pan part # 900786.091; lid part # 900779.901) or a hermetic aluminum pan (Pan part # 900793.901; lid part # 900794.901 (TA Instruments, New Castle Del. USA)). Non-solvated samples were loaded into standard pans and were sealed either by crimping for dry samples or press fitting for wet samples (such as slurries). Solvated samples (including hydrates) were loaded into hermetic pans and hermetically sealed. The sample pan was loaded into the Q1000 Differential Scanning Calorimeter, which is equipped with an autosampler, and a thermogram was obtained by individually heating the same using the control software at a rate of 10° C./minute from Tₘᵢₙ (typically 30° C.) to Tₘₐₓ (typically 300° C.) using an empty aluminum pan as a reference. Dry nitrogen (compressed nitrogen, grade 4.8 (BOC Gases, Murray Hill, N.J. USA)) was used as a sample purge gas and was set at a flow rate of 50 mL/minute. Thermal transitions were viewed and analyzed using the analysis software provided with the instrument.

### Thermogravimetric Analysis

Thermogravimetric analysis (TGA) of samples was performed using a Q500 Thermogravimetric Analyzer (TA Instruments, New Castle, Del., U.S.A.), which uses Advantage for QW-Series, version 1.0.0.78, Thermal Advantage Release 2.0 (2001 TA Instruments-Water LLC). In addition, the analysis software used was Universal Analysis 2000 for Windows 95/98/2000/NT, version 3.1E; Build 3.1.0.40 (2001 TA Instruments-Water LLC).

For the TGA experiments, the purge gas used was dry nitrogen, the balance purge was 40 mL/minute N₂, and the sample purge was 60 mL/minute N₂.

TGA was performed on the sample by placing a sample of the levodopa hydrochloride salt in a platinum pan. The starting temperature was typically 20 degrees C. with a heating rate of 10 degrees C./minute, and the ending temperature was 300 degrees C.

### Powder X-Ray Diffraction

Powder x-ray diffraction patterns were obtained using either a D/Max Rapid X-ray Diffractometer (Rigaku/MSC, The Woodlands, Tex., U.S.A.) or a Bruker D8 Discover with GADDS diffractometer (Bruker-AXS Inc., Madison, Wis., U.S.A).

The D/Max Rapid X-ray Diffractometer was equipped with a copper source (Cu/K_{α} 1.5406 Å), manual x-y stage, and 0.3 mm collimator. A sample was loaded into a 0.3 mm quartz capillary tube (Charles Supper Company, Natick, Mass., U.S.A.) by sectioning off the closed end of the tube and tapping the small, open end of the capillary tube into a bed of the powdered sample or into the sediment of a slurried sample. The loaded capillary tube was mounted in a holder that was placed and fitted into the x-y stage. A diffractogram was acquired using control software (RINT Rapid Control Software, Rigaku Rapid/XRD, version 1.0.0 (©1999 Rigaku Co.)) under ambient conditions at a power setting of 46 kV at 40 mA in transmission mode, while oscillating about the omega-axis from 0-5 degrees at 1 degree/second, and spinning about the phi-axis over 360 degrees at 2 degrees/second. The exposure time was 15 minutes unless otherwise specified.

The diffractogram obtained was integrated of 2-theta from 2-40 degrees and chi (1 segment) from 0-36 degrees at a step size of 0.02 degrees using the cyllnt utility in the RINT Rapid display software (RINT Rapid display software, version 1.18 (Rigaku/MSC)) provided by Rigaku with the instrument. The dark counts value was set to 8 as per the system calibration by Rigaku. No normalization or omega, chi, or phi offsets were used for the integration.

The Bruker D8 Discover with GADDS Diffractometer was equipped with a copper source (Cu/K_{α} 1.5406 Å), computer controlled x-y-z stage, a 0.5 mm collimator and a Hi-Star area detector. Samples were loaded into a proprietary sample holder by tapping the sample holder into a powder bed and arraying the holders into a 96 position block. The block was then loaded onto the x-y-z stage and the sample positions were entered into the software. A diffractogram was acquired using control software (GADDS—General Area Detector Diffraction System, (Bruker, version 4.1.14 (©1997-2003 Bruker-AXS.)) under ambient conditions at a power setting of 46 kV at 40 mA in reflectance mode. The exposure time was 5 minutes unless otherwise specified.

The diffractogram obtained was integrated of 2-theta from 2-40 degrees and chi (1 segment) from 0-36 degrees at a step size of 0.02 degrees using the GADDS software.

The relative intensity of peaks in a diffractogram is not necessarily a limitation of the PXRD pattern because peak intensity can vary from sample to sample, e.g., due to crystalline impurities. Further, the angles of each peak can vary by about +/−0.1 degrees, or by about +/−0.05. The entire pattern or most of the pattern peaks may also shift by about +/−0.1 degrees to about +/−0.2 degrees due to differences in calibration, settings, and other variations from instrument to instrument and from operator to operator. All reported PXRD peaks in the Figures, Examples, and elsewhere herein are reported with an error of about ±0.1 degrees 2-theta. Unless otherwise noted, all diffractograms are obtained at about room temperature (about 24 degrees C. to about 25 degrees C.).

### Raman Spectroscopy

The sample was either left in the glass vial in which it was processed or an aliquot of the sample was transferred to a glass slide. The glass vial or slide was positioned in the sample chamber. The measurement was made using an Almega™ Dispersive Raman (Almega™ Dispersive Raman, Thermo-Nicolet, 5225 Verona Road, Madison, Wis. 53711-4495) system fitted with a 785 nm laser source. The sample was manually brought into focus using the microscope portion of the apparatus with a 10× power objective (unless otherwise noted), thus directing the laser onto the surface of the sample. The spectrum was acquired using the parameters outlined in Table A. (Exposure times and number of exposures may vary; changes to parameters will be indicated for each acquisition.) The existence and magnitude of any expected error, such as experimental error, associated with the acquired Raman shift (cm⁻¹) of any one or more peaks within a Raman spectrum herein is known in the art and should be considered accordingly.

TABLE A
Raman Spectral acquisition parameters

| Parameter | Setting Used |
|---|---|
| Exposure time (s) | 2.0 |
| Number of exposures | 10 |
| Laser source wavelength (nm) | 785 |
| Laser power (%) | 100 |
| Aperture shape | pin hole |
| Aperture size (um) | 100 |
| Spectral range | 104-3428 |
| Grating position | Single |
| Temperature at acquisition (degrees C.) | about 24.0 |

### Example 2

### In Vitro Dissolution of Levodopa Hydrochloride

A dissolution study for a levodopa fast-dissolve tablet feasibility study was completed. All tablets were about 400 mg total weight and contained a target of 100 mg levodopa in four forms: levodopa free base, levodopa hydrochloride salt (from Example 1), levodopa free base jet milled (5-10 micrometers), and levodopa free base jet milled granulated with HPC-L solution. The filler for the tablet was Pharmatose DCL 14 (lactose). The dissolution was carried out in 200 mL of SGF (simulated gastric fluid) at 37 degrees C. using an overhead mixer at a speed of 50 rpm. The results, as shown in FIG. 5, are an average of N=2 trials and are normalized by the final concentration. As shown in FIG. 5, it took about twice as long (about 20 minutes versus 10 minutes) to realize >90% dissolved for the levodopa free base versus the levodopa hydrochloride salt. In the above described dissolution study, the SGF was prepared using the following preparation:

Dissolved 4 grams sodium chloride and 2 grams Triton X-100 in 2000 mL HPLC grade water. Added 1 N hydrochloric acid to the solution until the pH reached 2.0.

What is claimed is:

1. (−)-2-amino-3-(3,4-dihydroxyphenyl)propanoic acid hydrochloride.

2. The (−)-2-amino-3-(3,4-dihydroxyphenyl)propanoic acid hydrochloride of claim 1, wherein said hydrochloride is characterized by a powder X-ray diffraction pattern comprising peaks expressed in terms of 2-theta angles, and further wherein said X-ray diffraction pattern comprises peaks at 16.32, 18.82, and 19.51 degrees.

3. The (−)-2-amino-3-(3,4-dihydroxyphenyl)propanoic acid hydrochloride of claim 1, wherein said hydrochloride is characterized by a powder X-ray diffraction pattern comprising peaks expressed in terms of 2-theta angles, and further wherein said X-ray diffraction pattern comprises peaks at 21.65, 24.25, and 29.05 degrees.

4. The (−)-2-amino-3-(3,4-dihydroxyphenyl)propanoic acid hydrochloride of claim 1, wherein said hydrochloride is characterized by a powder X-ray diffraction pattern comprising peaks expressed in terms of 2-theta angles, and further wherein said X-ray diffraction pattern comprises peaks at 16.32 and 19.51 degrees.

5. The (−)-2-amino-3-(3,4-dihydroxyphenyl)propanoic acid hydrochloride of claim 1, wherein said hydrochloride is characterized by a powder X-ray diffraction pattern comprising peaks expressed in terms of 2-theta angles, and further wherein said X-ray diffraction pattern comprises peaks at 18.82 and 21.65 degrees.

6. The (−)-2-amino-3-(3,4-dihydroxyphenyl)propanoic acid hydrochloride of claim 1, wherein said hydrochloride is characterized by a powder X-ray diffraction pattern comprising peaks expressed in terms of 2-theta angles, and further wherein said X-ray diffraction pattern comprises a peak at 16.32 degrees.

7. The (−)-2-amino-3-(3,4-dihydroxyphenyl)propanoic acid hydrochloride of claim 1, wherein said hydrochloride is characterized by a DSC thermogram, and further wherein said DSC thermogram comprises an endothermic transition at about 195 degrees C.

8. The (−)-2-amino-3-(3,4-dihydroxyphenyl)propanoic acid hydrochloride of claim 1, wherein said hydrochloride is characterized by a DSC thermogram, and further wherein said DSC thermogram comprises an endothermic transition at about 236 degrees C.

9. The (−)-2-amino-3-(3,4-dihydroxyphenyl)propanoic acid hydrochloride of claim 1, wherein said hydrochloride reaches equal to or greater than about 90 percent in vitro dissolution at least about 1.25 times faster than that of levodopa free base.

10. The (−)-2-amino-3-(3,4-dihydroxyphenyl)propanoic acid hydrochloride of claim 1, wherein said hydrochloride reaches equal to or greater than about 90 percent in vitro dissolution at least about 1.5 times faster than that of levodopa free base.

11. The (−)-2-amino-3-(3,4-dihydroxyphenyl)propanoic acid hydrochloride of claim 1, wherein said hydrochloride reaches equal to or greater than about 90 percent in vitro dissolution at least about 2.0 times faster than that of levodopa free base.

12. The (−)-2-amino-3-(3,4-dihydroxyphenyl)propanoic acid hydrochloride of claim 1, wherein said hydrochloride reaches equal to or greater than about 90 percent in vitro dissolution at least about 2.5 times faster than that of levodopa free base.

13. The (−)-2-amino-3-(3,4-dihydroxyphenyl)propanoic acid hydrochloride of claim 1, wherein said hydrochloride is prepared as a pharmaceutical composition.

14. The (−)-2-amino-3-(3,4-dihydroxyphenyl)propanoic acid hydrochloride of claim 13, wherein said pharmaceutical composition further comprises a diluent, excipient, or carrier.

15. A process for the preparation of (−)-2-amino-3-(3,4-dihydroxyphenyl)propanoic acid hydrochloride, comprising mixing (−)-2-amino-3-(3,4-dihydroxyphenyl)propanoic acid with hydrochloric acid to form a mixture and allowing for precipitation of said (−)-2-amino-3-(3,4-dihydroxyphenyl)propanoic acid hydrochloride to occur.

16. The process of claim 15, further comprising adding a diluent, excipient, or carrier.

17. A method of treating Parkinson's disease, comprising administering an effective amount of the (−)-2-amino-3-(3,4-dihydroxyphenyl)propanoic acid hydrochloride of claim 1 to a mammal in need thereof.

18. The method of claim 17, wherein said mammal is a human.
